(19) United States
(12) Patent Application Publication
(10) Pub. No.: US 2021/0080398 A1
(43) Pub. Date: Mar. 18, 2021

(54) Sensor Including an Optical Fibre and Its Use in Wetness Monitoring

(71) Applicant: Oxford Caresense Ltd., Oxfordshire (GB)

(72) Inventors: Ejaz HUQ, Oxfordshire (GB); Mohamad Yasser AL AIOUBI, Oxfordshire (GB)

(21) Appl. No.: 16/772,404

(22) PCT Filed: Dec. 12, 2018
(86) PCT No.: PCT/GB2018/053611

§ 371 Date: Jun. 12, 2020

(30) Foreign Application Priority Data
Dec. 12, 2017 (GB) 1720685.5

(51) Int. Cl. G01N 21/81; A61F 13/42; G01N 21/77

(52) U.S. Cl. CPC G01N 21/81; G01N 21/7703; A61F 13/42

(57) ABSTRACT

A wetness monitor suitable for use in a diaper comprises an optical fibre having a core (11) and a cladding (12), borne on a substrate which is typically the outer layer (17) of the diaper. The fibre includes one or more discontinuities (13) in at least the cladding, and which may extend through the fibre; in that case the separate sections of the fibre are maintained in optical alignment. Ambient liquid enters the discontinuity preferentially, whereby the intensity of light passing through the core from a light source (14) to a light detector (15) is modified in the presence of liquid.

### FIELD OF THE INVENTION

This invention relates to a sensor including an optical fibre, and in particular to a device that can be used to measure wetness in an incontinence product, e.g. an adult or a baby diaper.

### BACKGROUND OF THE INVENTION

At present, the usual way to check that an incontinent patient's diaper does or does not need changing is to use nursing/caring effort to examine the patient. This involves changing the diaper, possibly unnecessarily, and expending staff time on the checking operation. The consequences of infrequent changing, as well as being uncomfortable and degrading for the patient, also carry significant medical risks. These risks are exacerbated when the patient is bed-bound. The risks of bed sores, other skin inflammations and infection are significant; they can be mitigated by proper hygiene and timely changing.

Various options have been proposed for monitoring soiling of incontinence products. US2013/0162403 describes a tag comprising a radio-frequency chip, an antenna, a memory element with electrical storage and output terminals, and a coating that becomes electrically conductive when wetted.

Other monitoring solutions involve an indicator that changes colour, positioned within the incontinence product, for example due to a change in pH when wetted with urine; see WO2010/049829.

US2010/164733 relates to a diaper sensor, using urine as an electrolyte.

US2012/165772 relates to a diaper sensor in which expansion changes or breaks an electrical circuit. This technology therefore has safety issues.

US2009/198202 relates to a magneto-elastic ribbon for use in a diaper. Wetness (or other chemical or biological analyte) is detected via a change in magneto-acoustic resonant frequency.

US2010/305530 relates to a fluid saturation sensor for monitoring wetness in sanitary towels. The sensor may be “resistive or capacitive”.

US2012/157949 (Kimberly-Clark) relates to an incontinence pad that has a visual indicator for wetness. The visual indicator is an “active barrier” that swells when wetted.

U.S. Pat. No. 8,978,452 relates to an electronic wetness sensor for an incontinence product that can be remotely checked via an RFID link. The sensor uses a frangible link, such that contact with urine causes the RF circuit to fail. Remote monitoring of the product is suggested.

### SUMMARY OF THE INVENTION

According to the present invention, an optical fibre of the type comprising a core and a cladding includes a discontinuity or gap into which liquid can migrate and thereby modify the intensity of light passing through the core. The gap may be formed in the cladding; the thus-exposed surface of the core may then be suitably modified. Alternatively, the gap may extend through the fibre, in which case the separate sections of the fibre are suitably borne on a substrate, in order to maintain their optical alignment.

It will be understood that, here and elsewhere in this specification, the use of the singular includes the plural. Thus, and is indeed preferred, there may be a plurality of gaps (or discontinuities).

Such a fibre can be used as a wetness sensor, and is suitable for use in an incontinence product or a baby diaper. The gap in the fibre provides a sensing region which is responsive to urine when the diaper is wetted. The two ends of the fibre may be connected to a light source and a light detector. The sensing region on the optical fibre may have a gap. The purpose of the gap is a) to allow leakage of the light when the diaper is in a dry state and b) ingress of liquid into the gap, drawn from the absorbent layer in the diaper, when the diaper is wet, using capillary action. The ingress of liquid into the gap allows more light to pass through the gap to the other side of the core, thereby indicating diaper wetting.

A sensor configured in this way is both accurate and reliable, and is capable of having a very low profile so that it is unobtrusive to a user when positioned within a diaper.

The method of the invention represents an improvement in monitoring of wetness in incontinence products. It allows for remote monitoring by a carer and can provide instant information, alerting the carer to the need to replace the incontinence product. The method is not dependent on pH and reduces waste compared to diapers without wetness monitoring and compared to previous wetness monitoring solutions.

Preferably, the method further comprises sending a signal by wireless communication to a remote monitoring unit. This allows a carer to have real-time information as to a user's requiring a fresh diaper and thus reduces patient discomfort by reducing the time a diaper is still in use when soiled.

### BRIEF DESCRIPTION OF THE DRAWINGS

The present invention will be described by way of example only with reference to the accompanying drawings, in which:

FIG. 1 is a simplified schematic of a typical diaper incorporating a wetness sensor in accordance with the invention;

FIG. 2 is a schematic of a typical diaper construction;

FIG. 3 is a schematic cross-section of a monitor of this invention;

FIG. 4 is an exploded schematic view of another monitor of this invention; and

FIGS. 5 to 12 are schematic views of different embodiments of optical fibres for use in the invention.

### DETAILED DESCRIPTION OF THE INVENTION

An incontinence product, such as a diaper, may comprise the wetness sensor of the invention. The wetness sensor of the invention is preferably incorporated into the diaper during manufacture of the diaper.

The diaper may comprise conventional materials. Generally, disposable diapers are made from absorbent material, e.g. wood/cellulose fibre and polyacrylate material on the inside to absorb the urine, and synthetic materials on the outside. Suitable such materials include polypropylene, polyester, and polyethylene for fitting and to prevent leaking. Incontinence diapers may comprise up to six layers. There are usually three essential layers: in order, a top liquid-permeable layer, a liquid-absorbent layer and a non-permeable layer. The absorbent layer is typically mostly fluff and a small quality of hydrogel. As liquid reaches the absorbent layer, the hydrogel soaks it up from the fluff and swells. Superabsorbent polymer is generally used as hydrogel.

In general, any suitable optical fibre may be used. The core may be of polymethyl methacrylate and the cladding of a fluorinated polymer. The fibre is preferably a step-indexed multimode or a multimode graded or a single mode fibre. A multimode fibre preferably has a diameter of up to 1 mm, e.g. 0.25 mm, and a smaller core diameter, e.g. 0.24 mm. A specific suitable fibre has a numerical aperture 0.5 and acceptable angle of 60 degrees with an attenuation @ 650 nm<0.3 dB/m and operating temperature −40+70 degrees Celsius.

A characteristic feature of this invention is a gap in the fibre. This structured region or sensing gap is typically in the region of 0.05 mm-1 mm in width and 0.25 mm-1 mm in height. The gap may be formed by cutting the fibre or, if primarily only in the cladding, using laser ablation, heated blade or a similar process. The purpose of the gap is to allow light to leak out. A significant portion of the light leaks out of the gap (e.g. approximately 30%) by transmission through or scattering by the material of a diaper, e.g. absorbent layer and the bottom substrate. The remainder (approximately 70%) of the light is transmitted to the other side of the fibre.

As the diaper gets wet, liquid is preferentially wicked into the gap. Liquid in the gap will help to increase the light throughput to the other side of the fibre, resulting in an increased signal at the light detector. Because the refractive index of air in the gap (n=1) is replaced by liquid (urine) with a refractive index in the region of 1.33, which is typically closer to the refractive index of the fibre core (1.49 in the case of polymethyl methacrylate), more light will pass through the core. Additionally, there will be some total internal reflection due to the water-air boundary.

In another embodiment, one or both of the fibre ends in the gap region may be angled to increase light coupling. The angled fibre approach provides enhancement of the effect of liquid bridging the gap. Not only is beam divergence reduced, but the walk-off of the beam due to exiting the angle facet is reduced as the liquid index is closer to the fibre core index. It can be thought of as amplifying the effect of the index change.

As indicated above, the gap may extend through the width of the fibre, such that the fibre is provided in at least two sections. Alternatively, a gap is formed in the cladding, and the fibre includes a roughened surface (for wicking water from the diaper absorbent layer). Roughening may be achieved by, for example, chemical etching, mechanical polishing or physical ablation (e.g. powder blasting). A small section of the fibre, e.g. 0.5-2 cm long, may be roughened. When a diaper including such a fibre is wet, liquid may be wicked either directly from the absorbent layer onto the roughened area or via a microchannel created in the vicinity of the structured region, which increases the quantity of light passing through the fibre, indicating diaper wetting.

In another embodiment, the exposed core region, following cladding removal by roughening, may be further etched to create micro-capillaries in the core, e.g. by a chemical process using a solution such as methyl isobutyl ketone (MIBK) and isopropyl alcohol (IPA). This may allow more light to leak out of the fibre when the diaper is in a dry state and more light coupled back into the core as the region is wetted, thus increasing the sensitivity of the sensor.

In another embodiment, the fibre is tapered along its length, in a sensing region. This requires the cladding to be removed. The critical angle when the fibre core is exposed to air is 42° (assuming refractive index n=1.49 for a polymethyl methacrylate core). This means that any light travelling such that its angle to the normal to the fibre wall/air interface is 42° or more will be reflected and travel down the fibre, essentially loss-free. If the cladding is in place this angle increases to 70°, so that only light that is nearer normal to the fibre axis is trapped. If the index outside the core is that of water, n=1.33, then the critical angle is 63°. If light travelling at an angle to the normal of 70° reaches a length of fibre that is bare, then the light is still confined, as it is already travelling at an angle>42° (i.e. 70°). If the fibre is now tapered at a suitable angle (i.e. not too steep, but steep enough that the light has a sufficient number of bounces in the sense region length) then, with each bounce, the ray will be travelling at a smaller angle to the normal between core surface and the outside environment. Eventually, it will reach the critical angle and escape. This will happen earlier if the fibre is clad with water, as the angle only has to reach 63°, not 42°. If the fibre diameter is 250 μm, a beam travelling at 70° to the normal will travel 686 μm before its first bounce, equivalent to 14 bounces along a 1 cm length. If the fibre tapers at 1°, then after this number of bounces it will have reduced its angle by 28°. It should be realised that the fibre will reduce in diameter by 23 μm.

The tapering of the fibre may be implemented in several ways, e.g. having a short length (1-2 mm) of tapering followed by a straight section and repeat of the same over a length of, e.g. 2 cm. A taper of 1° to 2° can be used. A taper provided over a short distance may be followed immediately by a flare (like a bowtie) followed by a straight section and repeat of this pattern over a distance e.g. 2 cm. The flare region will allow refocusing of the light into the core.

Gradually light will leak out of the taper. When the diaper is wet, liquid may be introduced to the tapered region via a micro channel (wicking liquid from the diaper absorbent layer) to increase the quantity of light going through the tapered region.

A product of the invention may comprise one or more microfluidic channels along a section on one or either side of the fibre, including the gap or the roughened region. The microfluidic channel wicks water from the diaper absorbent layer and drains it into the gap or the roughened region. Microchannels in a polymeric material will be essentially hydrophobic; the surface may be treated to render it hydrophilic.

The liquid wicking process is also assisted by pressure created by the diaper loading as the diaper absorbent layer gets wet.

Microchannels between the substrate and the fibre close to the structured region, may be formed using a tape or a similar material. The material is preferably hydrophilic. The channels are created between the tape edge and the fibre.

Microchannels may also be created in the substrate to draw liquid from the diaper absorbent layer into the structured region. Channels measuring up to 2 cm in length and up to 0.5 mm in width may be created in the substrate by a lithography process or physical ablation. In general, any substrate material can be used; however, to assist the wicking process a material having a hydrophilic surface is preferred.

Microchannels may also be created between two separate fibres on a flexible substrate, where the two fibres are in contact. To assist the wicking process, the fibres may be coated with a hydrophilic substance. In this case, a reflective surface may be provided, to reflect light from one fibre into the other.

In summary, the structured fibre has a core that is exposed to liquid that can be drawn from a diaper's absorbent layer such that the refractive index in the structured region is altered by the presence of the liquid. This is analogous to ‘repairing’ the roughened fibre cladding.

The optical fibre may comprise a plurality of sensing regions, spaced apart along the length of the fibre. This may facilitate selection of the trigger point at which a user or carer is alerted to soiling, such that a lesser or greater degree of soiling may prompt a change of diaper. The number of sensing regions is preferably from 2 to 6, e.g. 3. The distance between each optical sensor is preferably from 1 cm to 10 cm. A plurality of sensing regions, which may be accessed simultaneously, allows quantification of the liquid volume and therefore wetness level in the diaper (low, medium and full).

A sensor of the invention may comprise a single fibre, e.g. with a plurality of sensing regions, or a plurality of such fibres. The provision of more than one sensing fibre allows capture of liquid from a wider area. It may also allow for different possible orientations of the male organ, and may also be beneficial for incontinence assessment. Multi-strand sensors may utilise the same light source but separate photodetectors.

In a preferred embodiment, the sensor includes a reference fibre (without gaps) alongside the sensing fibre. This can be used to account for any change in signal caused by distortion of the diaper during use.

For use in a diaper, one end of the (or each) fibre may be connected to a light source and the other end to a light detector. It will often be convenient that a fibre is bent in a U-shape, so that its ends can be positioned at adjacent points on the waistband of a diaper. For this purpose, the fibre may be, for example, at least 20, e.g. e.g. 50 cm long.

Alternatively, an essentially straight single fibre may be used. A fibre coupler (e.g. 3 db) may then be used, where one end of the fibre may be mirrored or a separate reflective surface may be placed in front of the fibre end.

The fibre may be placed directly in a diaper and attached by, for example, ultrasonic welding. Alternatively, the fibre may be placed on a flexible substrate that is then used to form part of the diaper. The substrate material may be a polymer or a textile which can be either woven or non-woven. The substrate may be hydrophilic, to facilitate liquid wicking from the diaper absorbent layer.

The substrate dimension is typically in the form of a strip. It is preferably 1 cm-1.5 m in length, 1-15 mm in width and up to 1 mm thick. The fibre may be attached to the substrate by, for example, gluing or ultrasonic bonding.

If it extends through the fibre, the gap may be formed before or, preferably, after being positioned on a substrate. This allows the optical alignment of the separate sections of the fibre to be maintained.

In a preferred embodiment of the invention, the fibre (typically in a U-shape) is mounted between two plastics films that are laminated or otherwise bonded together. An aperture is provided in one film at a point corresponding to each sensing gap, and the wicking of liquid from a diaper through the aperture to the gap is preferably enhanced by placing an absorbent or wicking material, e.g. porous tissue, between the films. This material provides a path for liquid form a diaper, through the aperture(s), to the gap(s). This arrangement allows simple, low-cost manufacture of a unitary structure ready for placement in a diaper.

A sensor of the invention may be incorporated in a diaper or other incontinence product, preferably during manufacture. A strip incorporating a fibre of the invention may be placed inside the diaper's non-permeable layer such that the absorbent layer is in contact, directly or indirectly, with the fibre. The sensor strip is preferably placed in the central region, along the length of the non-permeable layer.

In an alternative embodiment, the sensor strip may be embedded within the absorbent layer such that one layer of the absorbent rests on the sensor layer and a second layer of absorbent is under the sensor strip. The absorbent layer rests non-uniformly on the sensor strip with air gaps between the fibre and the absorbent layer.

In another embodiment, the strip is placed under the permeable top layer, above the absorbent layer of the diaper, the sensor region facing the absorbent layer. This placement may facilitate rapid response to soiling by locating the sensor closest to where liquid enters the absorbent pad, whilst preventing direct contact with the skin of the user. The strip may have micro-perforations, to allow liquid ingress. In any case, the fibre must have access to the diaper absorbent layer, directly or indirectly.

In order to ensure that the fibre is not damaged during a diaper folding step in manufacture, the radius of curvature of the fibre is preferably not lower than 5 mm. This may be achieved by laying the fibre on the substrate in a particular configuration. One possibility is to fold the fibre, roughly in a figure of eight, such that when the diaper is folded the fibre can expand, without kinking.

To ensure ease of manufacture and convenient use, a connector may be placed at the sensor fibre ends. A complementary interrogator box has two fibres with a connector which is adapted to mate with the sensor fibre ends.

The fibre end or ends may be terminated at the edge of the substrate, and this edge inserted into a soft plastics ‘interrogator box’ comprising a LED, a photodetector, associated electronics and power supply. For ease of insertion, a small length of the substrate may be relatively thick, e.g. by providing an extra layer of the substrate material. Appropriate structuring of the box will allow alignment of the fibre ends with the LED and photodetectors.

In another embodiment, the fibre ends terminate in connector tubes. The mechanical tolerances of the tubes can provide consistent optical alignment. The interrogator box may be clipped or otherwise attached, e.g. using Velcro, to the diaper's waistband.

The light source may emit any infrared or visible wavelength of light. Preferably, the wavelength emitted is in the visible. Preferably, the light source is a light-emitting diode (LED). Alternatively, an organic light emitting diode (OLED) is used. An advantage of using OLEDs is that their manufacture can be low-cost, high throughput.

The light detector preferably comprises a photodiode (photodetector) or a phototransistor. Photodiodes and phototransistors use photons to generate electrons, thus enabling light detection. Photodiodes are readily available components that may be provided with a suitably low profile such that the interrogator box is unobtrusive to the user when placed on the waist in a diaper.

The interrogator preferably comprises a pulsed LED and a photodetector. Signals of order of μW will be received. This is well within the reach of a low-cost photodetector and amplifier. Phototransistors are also readily available components that may be provided in a low-profile form, with the additional benefit that the signal is amplified.

Such components provide sufficient signal to allow the use of a low-cost power consumption amplifier. Because of the signal level, high enough bandwidth (100 KHz or more) is possible, allowing short duty pulses of light for power saving which means battery life greater than 24 hours.

Preferably, the light emitted is pulsed. The modulation frequency may be varied if required for the circumstances of the individual user. The modulation frequency is preferably 1 pulse every 1 to 5 minutes. The length, or cycle, of each pulse is suitably of the microsecond scale. Using a pulsed light source allows the wetness sensor to have a low power consumption. The wetness sensor preferably operates with a supplied voltage of 5V or less, more preferably 3V or less.

A suitable box sends light down a fibre, and the received light is collected by a photodiode. A 3 dB coupler can be used to allow isolation between the outgoing light and the incoming, without the need for expensive, high-speed, power-hungry, time-division multiplexing.

The noise floor and bandwidth of the amplifier can allow a rapid enough LED pulse with no significant error in the detection signal. The LED is pulsed for two reasons: firstly, it reduces power consumption, to allow the interrogator to be powered by integral batteries for up to 24 hours, and secondly it affords immunity to offsets due to ambient light (DC light), or any other DC offsets affecting the reading. Nevertheless, ambient light should have little or no effect as the fibre in the diaper is in the dark, and the photodiode is enclosed in the interrogator box.

The system can ‘wake up’ by pulsing the LED, acquire the reading and transmit it to a monitor unit (Wi Fi etc.). If background light is a problem, repeat pulsing may be used to eliminate any DC offset.

The external control and monitoring unit may communicate wirelessly with a remote monitoring unit, for example a smartphone, laptop, pager or other device. This may allow a remote carer to be alerted to the need for a fresh diaper. Alternatively, the external control and monitoring unit may itself be capable of emitting an alarm when an alarm set point is reached.

Preferably, the electronic control and monitoring unit comprises an analogue-to-digital conversion unit; a plurality of indicators, for example one or more of audio, visual and tactile indicators; a wireless communication device; and a power source, for example a rechargeable coin cell providing up to 3.3 volts.

Preferably, the external control and monitoring unit has capability for individual patient identification provided by an RFID chip. This may facilitate remote monitoring of wetness in multiple diapers simultaneously. Preferably, in the dry state, the photocurrent will be below a minimum threshold current such that no alarm is triggered.

The external control and monitoring unit is capable of electrical connection with the wetness sensor. Preferably, the external control and monitoring unit is provided with a clip or Velcro® type mechanism for convenient attachment to a diaper, for example at a waistband. In another preferred embodiment, the external control and monitoring unit is provided with a cable to connect with the wetness sensor. In this configuration, the external control and monitoring unit may be positioned or held in a location more comfortable for the wearer of, e.g. a diaper.

Preferably, the external control and monitoring unit is reusable and detachable from a diaper. Preferably, each individual user is provided with a unique external control and monitoring unit.

One benefit of the wetness sensor of the present invention is that the wetness trigger point for an alarm can be varied according to user needs. For example, to improve patient comfort it may be desired to trigger an alarm when the incontinence product is partially loaded, rather than leaving it until fully loaded. This is possible because a plurality of gaps can provide a linear array of sensing regions which progressively get activated as the diaper is getting more wet. This can be contrasted with known wetness sensors that rely on a binary change of state and so are not tuneable.

Further, a plurality of sensing regions may be positioned and spaced apart by a predetermined distance such that the time the liquid takes to diffuse to the sensor can be monitored. The relative times at which each of a plurality is sensors is activated is indicative of a certain volume of liquid.

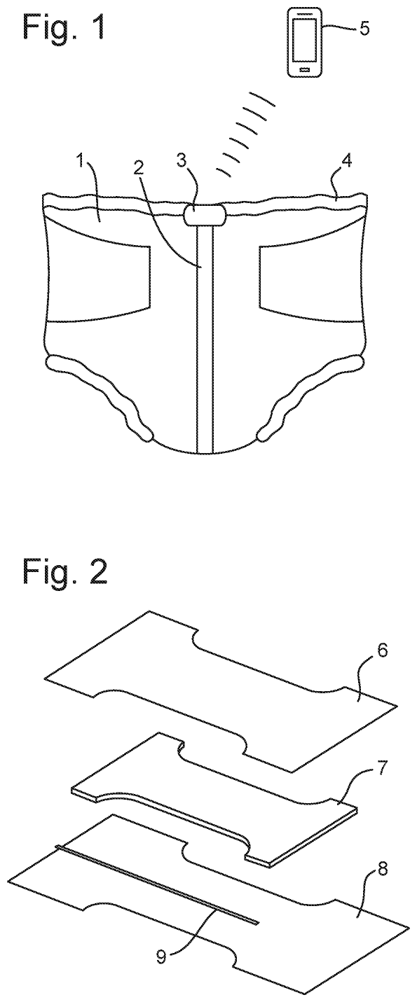

Referring now to the drawings in greater detail, FIG. 1 shows a diaper 1 that incorporates a wetness sensor 2 according to the present invention. The wetness sensor 2 may be positioned on or off-centre relative to the line of symmetry running from the front to the back of the diaper 1. An external monitoring 3 unit is clipped on to a waistband 4 of the diaper 1. The external control and monitoring unit 3 completes optical and electrical connection to the ends of the wetness sensor 2. The wetness sensor 2 extends down from the waist band 4 of the diaper 1 towards the crotch area such that the optical sensor may be positioned in the region most likely to be soiled.

FIG. 1 also shows wireless communication between the external monitoring 3 unit and a remote monitoring unit 5. The remote monitoring unit 5 enables a carer to be alerted to soiling of the diaper 1 without manually checking.

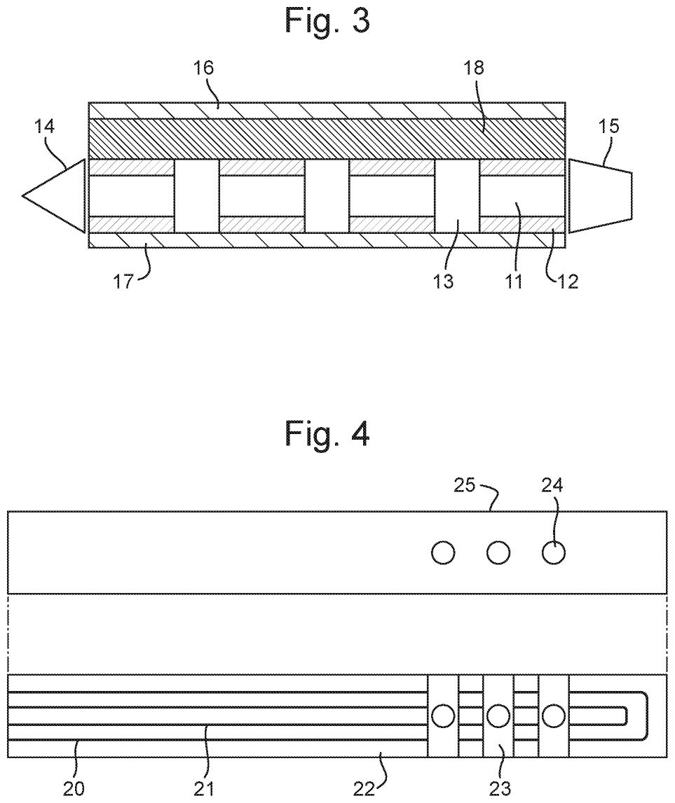

FIG. 2 shows the internal construction of a diaper. There are up to six layers in a diaper. The three basic layers are shown, including a top permeable layer 6, an absorbent layer 7, and a non-permeable back layer 8. Liquid (urine) passed by the patient goes through the permeable layer and reaches the absorbent layer (typically comprising fluff and hydrogel) where it soaks in. The non-permeable layer 8 stops leakage of the liquid outside the diaper. A structured optical fibre 9 is borne on the layer 8.

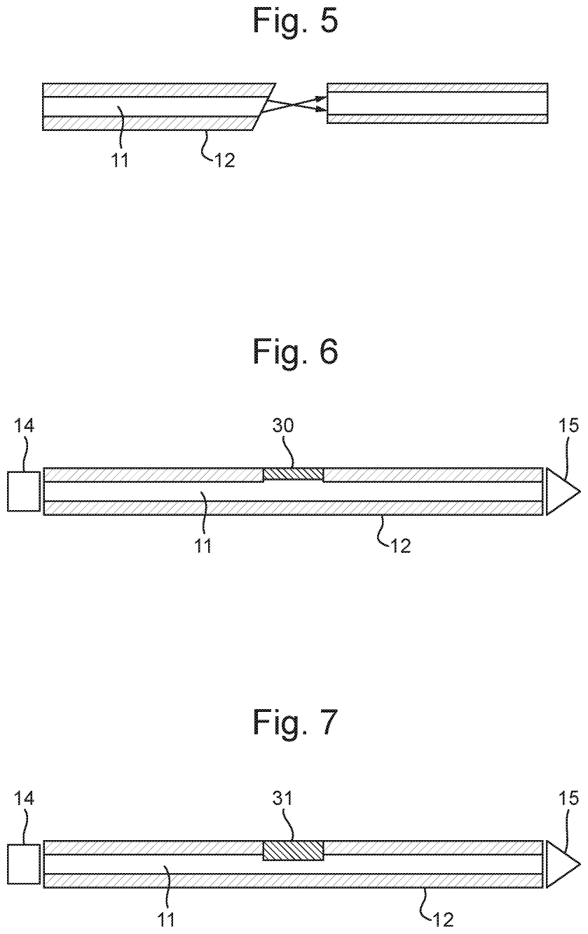

FIG. 3 shows a diaper including an optical fibre comprising a core 11 and a cladding 12, and including three gaps 13. A light source 14 and a light detector are provided. The diaper comprises an inner (permeable) layer 16, an outer (non-permeable) layer 17 on which the pieces of the fibre are borne, and an absorbent layer 18.

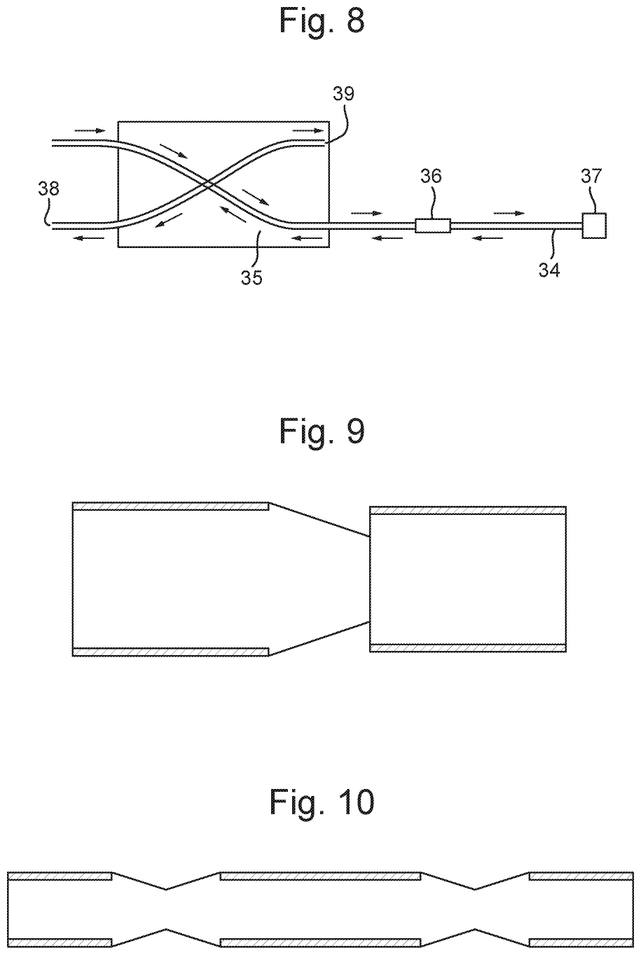

FIG. 4 shows a sensor strip in which a sensing fibre 20 and a reference fibre 21 are borne on a substrate 22. The sensing fibre 20 includes three gaps corresponding to the positions of three pieces of absorbent material 23 and apertures 24 in a top strip 25. Bonding or laminating of the strips 22 and 25 provides a unitary product suitable for placement in a diaper.

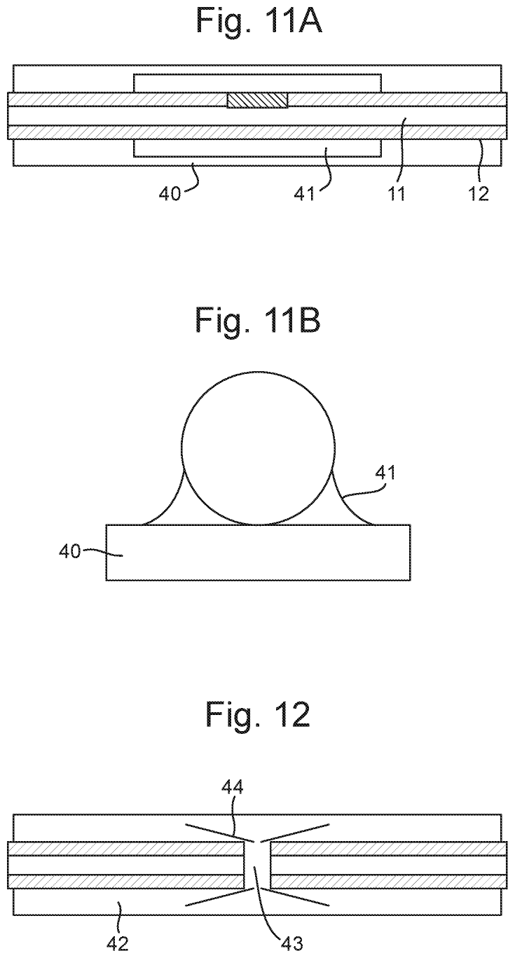

FIG. 5 shows that, by structuring the end of the fibre where the cut is made, it is possible to focus the leaked light into the core across the gap. As opposed to a straight cut the angled cut, preferably but not limited to 45°, will increase the light coupling.

FIG. 6 shows an optical fibre for use in the invention, including a microstructured region 30 in the cladding. The purpose of the roughened region is to allow light to leak out of the fibre when the diaper is in a dry state and causing the signal to drop at the detector. Roughening may be achieved by a number of processes including but not limited to wet chemical etching, dry etching (plasma process), laser ablation etc. When the diaper is wet, liquid from the absorbent layer of a diaper may be wicked either directly or assisted by micro channels (not shown). The ingress of the liquid in the roughened region will increase the light coupling and there will be an increased signal at the detector.

FIG. 7 is similar to FIG. 6 except that the roughening (micro texturing) in region 31 is extended into the core region, past the cladding so that higher leakage is possible when the diaper is wet and conversely increased signal is achieved when the diaper is in a wetted state and liquid is wicked either directly by the roughened region or assisted by micro channels (not shown).

FIG. 8 shows the use of a fibre 34 with an optical coupler 35. One arm of the construction has a sensing region 36 to leak and receive light as when the absorbent layer is dry and wet, respectively. The length of fibre is immediately halved, because there is no ‘U’ turn in the fibre, thus saving cost, and the effective length of the sensing regions is doubled, thus giving higher sensitivity. This may be achieved by adding a mirrored surface at the remote end of the fibre or by using a separate micro mirror 37. In a 4-port arrangement, if light is fed into the fibre 34, then it will for example of a 50:50 coupler exit at points 38 and 39. If fibre 34 is the sense fibre and is terminated by a reflector then some light will return and 50% of it will be available at 38 (in the opposite directions of the arrows shown). The important feature here is that no light will emerge unless is has been reflected by the sense fibre.

FIG. 9 shows a tapering in a section of a fibre, after removing the cladding and exposing the core. This may be achieved by a wet chemical or a dry process such as plasma etching or laser ablation using a mask. Using a thin fibre, the angle is critically controlled such that enough of the core is left behind for the fibre not to become too fragile whilst the cladding is removed from a sufficient region for the light to leak out causing a significant change in the signal. The angle should be of the order of 1° to 2°. This will allow bulk of the core to remain whilst exposing the cladding to the surrounding region. A 1 mm tapered region in the fibre is sufficient to allow perceptible change. As wicked into the tapered region the signal should recover significantly.

FIG. 10 shows that, to enhance the sensitivity of the tapering there may be several taper-flare and straight regions over a section of the fibre. The purpose of the flares is to focus the light more efficiently into the core.

FIGS. 11A and 11B show an optical fibre on a flexible substrate 40. A flexible tape 41 of a polymeric material covers a region of the fibre including the structured region. The purpose of the tape is to create micro channels in the vicinity of the fibre, to assist in the liquid wicking process. The micro channels run along the wall of the fibre. The tape which has an adhesive-coated surface may also be used to hold the fibre on the substrate. Depending on how the tape is wrapped, the channels may have a width in the region of 0.05 mm to 0.5 mm. The channels lead up to the gap (not shown) or roughened (not shown) region of the fibre where the liquid is delivered.

FIG. 12 shows an optical fibre on a substrate 42, the fibre including a structured gap 43. Microchannels 44 are created in the flexible substrate. Here and elsewhere, it will be understood that there may be more than one such region.

The following Examples illustrate the invention.

### Example 1

Step-indexed unjacketed multimode optical fibre (fibre diameter 0.25 mm and core diameter 0.24 mm) was used. Other characteristics of the fibre include: core material: polymethyl methacrylate, cladding: fluorinated polymer, numerical aperture: 0.50, acceptance angle: 60 degrees, attenuation @ 650 nm<0.3 dB/m, minimum bending radius: <9 mm and operating temperature −40+70 degrees Celsius.

A 40 cm length of this optical fibre was placed on a 0.1 mm thick flexible substrate. One end was connected to an LED and the other end to a lux meter (photodetector). When the LED was switched on, the lux meter reading was 330 lux. Then three cuts were made in the fibre using a heated blade. The signal in the lux meter dropped to 100 lux after the first cut, 33 lux after the second cut, and 9 lux after the third cut.

The optical fibre-based wetness sensor was then placed inside an incontinence diaper (commercially obtained—TENA), to provide the arrangement shown in FIG. 3. The diaper was then put on a mannequin. One end of the fibre was connected to a light source (LED) and the other end to the lux meter.

A 5 mm (internal diameter) plastic tube was inserted inside the diaper, ending in the inner permeable layer. The other end of the tube was connected to a mounted plastics bottle filled with saline water (substitute for urine). An adjustable-valve mechanism along the tube allowed control of liquid flow inside the diaper.

Water was introduced inside the diaper at regular intervals (10 ml/min), to a total of 600 ml water. However, the signal in the lux meter remained unchanged at 9 lux. 19 Lux was recorded at 200 ml, corresponding to the first cut, then 28 lux at 340 ml for the second cut, and finally 33 lux for the third cut at 590 ml.

Then microfluidic channels were created in the sensor. This was achieved by wrapping small pieces of plastic tapes on sections of the fibre with gaps.

When water was introduced inside the diaper, the microfluidic channels wicked water from the absorbent layer and filled the gaps in the fibre. Due to the particular placement of the sensor strip inside the diaper, the gaps were progressively filled with water, as water spread across the absorbent layer. This caused the signal in the lux meter to rise gradually. At the end of the experiment, 600 ml of water was introduced inside the diaper, and all three gaps were filled. The effect of having water inside the gaps equated to reintroduction of the cladding, causing more and more light to be coupled to the fibre and the increase in signal. The progressive increase in the signal is also indicative of the quantity of water inside the diaper.

Electronics and associated software were then developed to remotely monitor the water volume on a mobile app. via Bluetooth and WiFi.

### Example 2

The sensor used in this experiment is as shown in FIG. 4.

Two step-indexed unjacketed multimode optical fibres of the same type as in Example 1 were used, one acting at the sensing fibre and the other as a reference fibre. A thin (100 micron) transparent plastics strip, 30 cm long and 16 mm wide, was used as the substrate.

The sensing fibre had a length of 59 cm and the reference 57 cm; both were turned into a U-shape, and placed on a substrate using a thin layer of glue. Three pieces of tissue paper (absorbent layer) were put along the width of the fibre sensing regions, using a thin layer of glue to hold them in place.

A second transparent plastic strip having the same dimensions as the substrate was used as the top sheet. The top sheet had three holes covering the three pieces of absorber. The top sheet was then put on the substrate (completely aligned) and the two strips (substrate and top sheet) were then laminated. Following lamination, three equidistant cuts were made in the sensing fibre using a hot blade.

One end of the sensing fibre and one end of the reference fibre were placed in front of a LED, and the other two ends of the fibres (sensing and reference) were put in front of two separate photodiodes (detectors).

Before any cut was made in the sensing fibre, the signal at the photodetector recorded 1000 mV. After the first cut, the signal dropped from 1000 mV to 580 mV, after the second cut it dropped to 200 mV, and after the third cut to 50 mV.

For the reference fibre (without cuts), 800 mV was recorded at the photodetector. The difference between the initial value of 1000 mV for the sensing fibre and 800 mV for the reference fibre was due to some misalignment of the reference fibre.

A commercially procured incontinence diaper was used for the wetness test. The diaper was cut open from inside to insert the laminated sensor strip on top of the non-permeable layer (therefore under the diaper absorbent layer). The diaper (with the sensor strip) was put on a mannequin.

The sensor strip was connected to an electronics box comprising a LED, two photodetectors, signal amplifier and a power supply.

A plastic tube for introducing saline water (substituting for urine) was inserted inside the diaper, and the other end of the tube connected to a water reservoir. Using a control valve allowed control of liquid flow inside the nappy. This was an accelerated test. As the water filled up the first cut (gap in the fibre), the signal after 500 seconds at the photodetector recorded 100 mV (the effect of having water inside the gaps equated to reintroduction of the cladding, causing more and more light to be coupled to the fibre and the increase in signal). The water volume inside the diaper at this point was 125 ml. After 1100 seconds, when water filled up the second cut, the photodetector recorded 200 mV and the water volume inside the diaper was 250 ml. Finally, after 2200 seconds, when water filled the third cut, 400 mV was recorded. At this point the water volume inside the diaper was 625 ml. All through this experiment, for the reference fibre, the photodetector recorded a constant 800 mV, as was expected.

Electronics and associated software have been developed to remotely monitor the water volume on a mobile app, via Bluetooth and WiFi.

1. A wetness monitor comprising an optical fibre borne on a substrate, wherein the fibre comprises a core and a cladding and has a discontinuity in at least the cladding, provided that if the discontinuity extends through the fibre then separate sections of the fibre created by the discontinuity are in optical alignment, and wherein the structure is adapted such that ambient liquid preferentially enters the discontinuity, whereby the intensity of light passing through the core is modified in the presence of liquid.

2. The monitor according to claim 1, wherein the substrate or the surface of the core comprises microchannels.

3. The monitor according to claim 1, wherein the surface of the core is modified by ablation or etching.

4. The monitor according to claim 1, wherein the discontinuity extends through the fibre.

5. The monitor according to claim 1, wherein the discontinuity is 0.025 to 0.5 mm wide.

6. The monitor according to claim 1, wherein the discontinuity is 0.025 to 0.5 mm deep.

7. The monitor according to claim 1, which additionally comprises a reference fibre, wherein the reference fibre is an optical fibre without discontinuities.

8. A diaper comprising a monitor according to claim 1.

9. A method of detecting wetness in a diaper according to claim 8, which comprises passing light through the fibre, and observing a change in the intensity of the light.
